(19) United States
(12) Patent Application Publication
(10) Pub. No.: US 2007/0179629 A1
(43) Pub. Date: Aug. 2, 2007

(54) Acetabular cup assembly and cup revising method

(76) Inventor: Stephen B. Murphy, Winchester, MA (US)

Correspondence Address: CESARI AND MCKENNA, LLP, 88 BLACK FALCON AVENUE, BOSTON, MA, 02210, US

(21) Appl. No.: 11/344,408

(22) Filed: Jan. 31, 2006

(51) Int. Cl. A61F 2/34

(52) U.S. Cl. 623/22.28

(57) ABSTRACT

An acetabular cup assembly includes a cup-like liner with a rim defining a first plane, an inner surface defining a hemispherical socket with a center of curvature close to the first plane and an outer frustoconical seating surface having a first taper and extending to the rim. The assembly also includes a sleeve having inner and outer frustoconical surfaces and a central axis and surrounding the liner. The inner surface of the sleeve has the same taper as the seating surface so that the sleeve wedges against said seating surface to fix the position of said center of curvature relative to the sleeve. The outer surface of the sleeve has a selected second taper which matches that of the seat surface of an acetabular cup shell in which the sleeve is seated. A method of revising an implanted acetabular cup assembly is also disclosed.

This invention relates to hip replacements. It relates more particularly to the acetabular cup component of such a replacement and to a cup assembly and a method of revising the cup in the event of failure following the original implantation thereof.

### BACKGROUND OF THE INVENTION

Field of the Invention

Total hip arthroplasty (THA) is an effective surgical procedure for the relief of pain and the restoration of function of a diseased hip. Successful THA has contributed to enhanced mobility and comfortable independent living for people who would otherwise be substantially disabled. As shown in FIG. 1, in total hip replacement surgery the surgeon replaces a patient's diseased hip joint with an implant 10 consisting of a femoral stem 12 which is inserted into the femur F and an acetabular cup 14 which is anchored in the patient's pelvis L. The upper end of the stem 12 carries a head or ball-type bearing 12a. The head seats in a hemispherical socket 14a defined by the cup 14 so that the stem can swivel relative to the cup about the center of curvature C of socket 14a in the manner of a natural hip joint such as the one indicated at H in FIG. 1. In some total hip replacements, the femoral head 12a and the surface of the socket 14a are of metal so that those replacements have metal-on-metal bearing surfaces. Other known replacements employ acetabular cups comprising a metal outer shell fitted with an interior liner or bearing of a wear-resistant plastic, e.g. polyethylene, so that the stem head articulates against the plastic material. A consequence of such metal-on-metal and metal-on-plastic articulation is surface wear which liberates metal and/or plastic wear debris. Today, osteolysis as a result of wear debris is a major cause of long term failure of primary THA.

The increasing volume of THA performed in younger patients, in conjunction with a greater patient longevity has raised expectations of implant survivorship beyond that expected of traditional metal-on-metal and metal-on-plastic bearings. Therefore, enhancing these articulations has been aggressively pursued over the years. To that end, ceramic-on-ceramic hip replacement bearings have come into use and offer the opportunity to substantially eliminate wear debris. A typical ceramic-on-ceramic hip replacement comprises a femoral stem 14 similar to the one shown in FIG. 1, but having a modular head 12a of a ceramic material and an acetabular cup of the type shown in FIG. 2. As seen there, the cup 14 is composed of a metal outer shell 16 fitted with an inner ceramic liner 18 which forms the socket 14a for the femoral head which will rotate about the socket's center of curvature C. A ceramic commonly used for this purpose is alumina.

A critical element of such a cup is the taper junction of the liner and the outer shell. More particularly, the opposing surfaces 18a and 16a of the liner and shell, respectively, are frustoconical with matching tapers, e.g. 18°, so that the liner wedges into the shell in correct alignment therewith. The advantages of using ceramic components for the articulation in primary THA the fact that their wear debris particles are less bio-reactive than other wear debris particles and they have greater resistance to scratching and wear.

While all articulations in THA, including a ceramic-ceramic articulation, generate wear debris, the amount of cytokines in aseptically loose ceramic-on-ceramic THA components is significantly less than that found in the presence of metal-on-plastic debris. Therefore, based on the particular size, volume and relatively less bio-reactive properties, the incidence of wear debris inducted osteolysis in THA with ceramic-on-ceramic articulations may be less than that seen with other bearings.

One of the main drawbacks of THA is the risk of implant failure necessitating a so-called revision, i.e. the necessity to replace one or both of the hip replacement components (i.e. femoral stem and/or acetabular cup) following the original implantation. The need for such a revision THA may be due to any one of a variety of considerations such as infection, implant loosening, wear, component malposition, osteolysis, or even a defect in a replacement prosthesis.

A major problem with the acetabular cup component of a ceramic-on-ceramic hip replacement has to do with revision. More particularly, ceramic liners are quite fragile and any imperfection in the taper junction between the liner and the shell can result in fracture of the new liner. In other words, the seat surface of the shell may have little nicks or burrs that create stress risers that increase the risk of ceramic fracture of the liner. Resultantly, implant manufacturers recommend against inserting a new ceramic liner into an existing shell during revision THA for any reason.

Given these circumstances, a surgeon currently has four options, all of which have disadvantages, namely:
1.) insert a new ceramic liner 18 into an existing metal outer shell 16 against the recommendations of the manufacturer, which option risks fracture of the new liner and allegations of malpractice.
2.) insert a new metal shell 16 that allows insertion of a new ceramic liner 18 which is a poor option because it involves a much more extensive and risky operation to remove a perfectly positioned and fixed shell that has already had boney in-growth into its posterior surface;
3.) insert a new metal liner into an existing shell 16 which has two disadvantages, namely the metal-on-metal bearing surfaces produce metal ion debris which can circulate throughout the body and cause an allergic reaction, and the preexisting ceramic debris particles which are harder than metal can potentially cause scratching of the bearing and third-body debris, both of which can cause accelerated wear, and
4.) insert a new plastic liner into the shell 16 which is even worse than the previous option because third-body wear and scratching of the bearing surfaces will cause even more accelerated wear if plastic is used.

### SUMMARY OF THE INVENTION

Accordingly it is an object of the present invention to provide an adapter sleeve to improve the performance of the acetabular cup component of a total hip replacement and which may be used in both primary and revision total hip replacement circumstances.

Another object of the invention is to provide such an adapter sleeve which can be used to alter the position of the center of rotation of the femoral head of a total hip replacement.

Another object of the invention is to provide an adapter sleeve of this type which facilitates inserting a new ceramic liner in an already implanted shell of an acetabular cup assembly during either primary or revision hip replacement surgery.

Yet another object of the invention is to provide an adapter sleeve which allows the shell of an acetabular cup to accommodate liners of different sizes and articulation characteristics.

A further object of the invention is to provide an acetabular cup assembly incorporating an adapter sleeve having one or more of the above characteristics.

Still another object of the invention is to provide a method of revising the acetabular cup component of a hip replacement to alter the performance of that replacement.

Other objects will, in part, be obvious and will, in part, appear hereinafter.

The invention accordingly comprises the several steps and the relation of one or more of such steps with respect to each of the others and the apparatus embodying the features of construction, combination of elements and arrangement of parts which are adapted to effect such steps, all as exemplified in the following detailed description, and the scope of the invention will be indicated in the claims.

Briefly, my acetabular cup assembly comprises a more or less conventional shell having a generally hemispherical outer wall extending to a rim that defines a first plane and an inner wall defining a frustoconical seat extending into the shell from the first plane, the seat having a selected maximum diameter in that plane and a selected first taper. The cup also includes a more or less conventional cup-like liner having a rim defining a second plane, an outer wall defining a frustoconical outer seating surface that has a maximum diameter in the second plane and a selected second taper, said liner also having an inner wall defining a hemispherical socket with a center of curvature at or near the second plane.

Normally when matching a liner to the shell of an acetabular cup, a liner is selected whose seating surface has the same maximum diameter and taper as the seat of the shell so that the former can wedge tightly against the latter forming a taper junction. In contrast to that, the liner of my cup assembly is intentionally mismatched to be smaller than usual to allow the interposition between the liner and the shell of a frustoconical adapter sleeve having an outer surface that seats flush against the seat of the shell and an inner surface which is flush with the seating surface of the liner. In other words, the taper is of the outer surface of the sleeve matches that of the seat of the shell and the taper of the inner surface of the sleeve matches that of the seating surface of the liner.

As will be described in more detail later, the sleeve may be designed and dimensioned so that the liner and the sleeve together emulate a larger conventional liner that is matched to an existing shell. Alternatively, the sleeve may be designed and dimensioned so that the center of the liner socket is shifted perpendicular and/or parallel to the plane of the shell rim, i.e. said first plane. In this way, by proper sleeve selection during revision surgery, a defective or worn liner may be replaced by a new liner/sleeve combination that gives the hip replacement different spatial and/or articulation characteristics compared to the original replacement to correct some deficiency, e.g. an unstable hip due to soft tissue laxity, an unstable hip due to acetabular component malposition, or a hip where the neck of the prosthesis impingement against the prosthetic acetabulum due to component malposition.

Thus to perform a revision hip replacement according to my procedure, the original replacement is surgically accessed and the femoral component thereof is separated from the acetabular cup, the existing liner is removed from the shell of that cup while the shell remains in place. The shell is cleaned and a new liner/sleeve combination is selected which when inserted into the shell will position the center of the replacement liner socket at the desired location relative to the existing implanted shell. Depending upon the aforesaid selection, that location may be the same as it was in the original cup or the center of the revision may be offset from the original center, or the liner itself may be tilted relative to the shell to change the orientation of the socket to correct for some impingement or dislocation of the original hip replacement.

Although this invention applies principally to revision THR, it may also apply to primary THR. This may occur in a situation where the surgeon is happy with the cup position relative to the pelvic bone, but is unhappy with the cup position relative to the way that the hip works. That is, an implanted cup could be misorientated in any direction. Clinically, these orientations are termed “adduction/abduction” in the coronal plane, “flexion/extension” in the sagittal plane, and “anteversion/retroversion” in the transaxial plane. In those situations, the present adapter sleeve could be used to increase the offset or to change the anteversion of the articulation of the cup while leaving the metal shell as is.

The use of my adapter sleeve and cup assembly should facilitate both primary and revision hip replacements, particularly those employing acetabular cups with ceramic liners, without unnecessarily complicating the surgical procedure or causing undo discomfort to the patient.

### BRIEF DESCRIPTION OF THE DRAWINGS

For a fuller understanding of the nature and objects of the invention, reference should be made to the following detailed description taken in connection with the accompanying drawings, in which:

FIG. 1, already described, is a diagrammatic view of a pelvis with a natural hip and a total hip replacement which includes an acetabular cup;

FIG. 2, already described, is a cross sectional view on a larger scale of a conventional acetabular cup having a ceramic liner;

FIG. 3 is a exploded perspective view showing an acetabular cup assembly incorporating my invention;

FIG. 4 is a cross sectional view of the FIG. 3 assembly, as assembled;

FIG. 5 is a view similar to FIG. 4 showing a cup revision whose socket has a center of curvature which is offset from that of the original cup, and

FIG. 6 is a similar view of a cup revision whose socket is tilted with respect to the original socket.

### DETAILED DESCRIPTION OF THE PREFERRED EMBODIMENTS

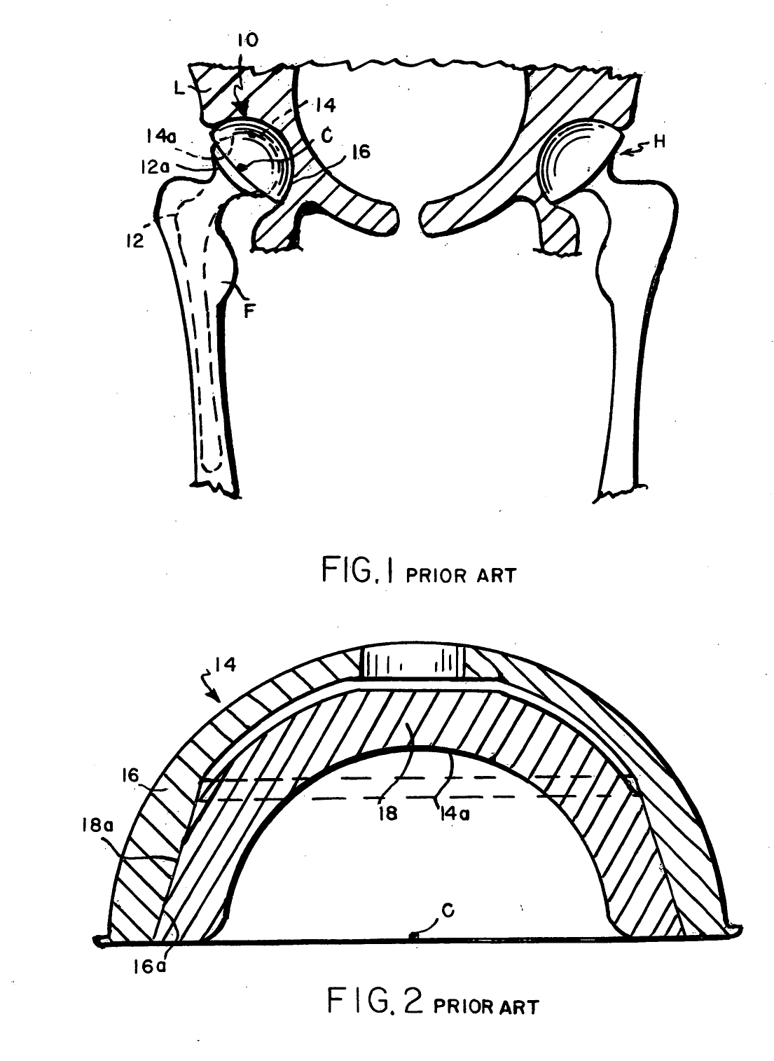

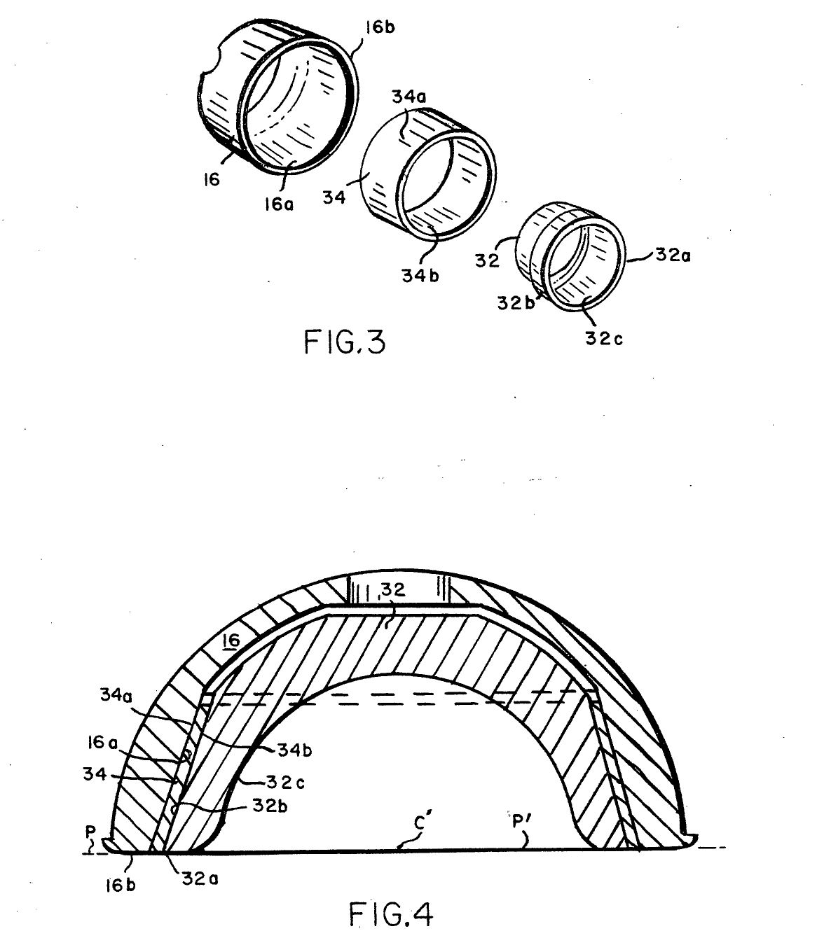

Referring to FIGS. 3 and 4 of the drawings, my acetabular cup assembly includes an already implanted acetabular cup shell such as shell 16 shown in FIGS. 1 and 2. Typically, shell 16 is of a biocompatible metal such as titanium and has a circular rim 16b defining a plane P. The frustoconical inner surface 16a of shell 16 extends into the shell from that rim and has a selected conical taper, e.g. 18°

The assembly also includes a cup-like liner 32 preferably of a ceramic such as alumina or a combination of alumina and zirconia with a circular rim 32a defining a plane P′, a frustoconical outer seating surface 32b extending from said rim toward the closed end of the liner and a hemispherical inner surface defining a socket 32c having a center of curvature C usually located within the liner at the plane P′.

The remaining component of the assembly is a frustoconical adjuster sleeve 34 having a frustoconical outer surface 34a and a frustoconical inner surface 34b. The adjuster sleeve 34 is of biocompatible metal, e.g. titanium alloy or cobalt chromium alloy. It is shaped and dimensioned to receive liner 32 so that the tapered seating surface 32b of the liner is flush against the inner surface 34b of the sleeve. Also, sleeve 44 is adapted to seat in shell 16 so that the tapered outer surface 34a of the sleeve is flush against the frustoconical seat surface 16a of the shell. In other words, the sleeve surfaces 34a and 34b are equidistant and parallel to one another and the two tapers are the same, i.e. the spacing of the inner and outer surfaces of the sleeve is uniform around the sleeve axis and the surfaces 34a and 34b have the same axis of curvature. With this configuration, the inner diameter of the shell is equal to the outer diameter of the liner plus twice the wall thickness of the sleeve. When assembly 30 is so seated in shell 16, the mating frustoconical surfaces of the shell, sleeve and liner are wedged against each other so that the plane P′ defined by the liner rim 32a is more or less coincident with the plane P defined by the shell and the center of curvature C′ of the liner socket is centered in shell 16 substantially at the planes P, P′, all as shown in FIG. 4.

In the cup revision assembly illustrated in FIGS. 3 and 4, all the tapered surfaces 16a, 32b, 34a and 34b have a taper of about 18° which is a standard in the industry. However, the taper could just as well be greater or less than that angle so long as it produces a tight wedging engagement of those surfaces. Desirably, the width of sleeve 34 is in the order of one third the diameter thereof so that the sleeve contacts the shell and liner over relatively large surface areas to assure a firm wedging engagement of those parts and proper mechanical support for the ceramic acetabular liner. Preferably also to accommodate tolerances, the seat surface 16a of the shell is slightly wider than the sleeve 34 as shown in FIG. 4 to assure that the sleeve can seat properly in shell 16.

Still referring to FIGS. 3 and 4, due to the presence of the adjuster sleeve 34, the liner 32 is obviously smaller than a liner that would normally fit in the shell 16, i.e. liner 18 shown in FIG. 2, in order to accommodate the wall thickness of the adjuster sleeve 34 which should be at least 2-3 mm. However, ceramic liners are presently available in a variety of sizes as shown by the following Table 1, with more sizes becoming available all the time as new and stronger ceramic materials come into use.

TABLE 1

| | | |
|---|---|---|
| 28/35 | 32/41 | 32/48 |
| 28/37 | 28/44 | 36/48 |
| 28/39 | 32/44 | 28/52 |
| 32/39 | 36/44 | 32/52 |
| 28/41 | 28/48 | 36/52 |

The first number of each entry in Table 1 is the internal diameter in millimeters of the ceramic acetabular liner 32 and the second number is the maximum diameter in millimeters of the rim of the ceramic acetabular liner 32a. All of these particular liners have a taper of 18°.

Thus, for example, if a 28/39 liner 18 is in place in the acetabular cup shown in FIGS. 1 and 2 and a revision becomes necessary, a 35/39 adapter sleeve 34 could be seated in the existing shell 16 and a new 28/35 ceramic liner could be inserted into the adapter sleeve 34. With those dimensions, the revision may include a sleeve 34 with a wall thickness of 2 mm, i.e. ½ (39−35).

Similarly, if a 32/41 liner 18 is in place in the cup shown in FIG. 2, a 37/41 adapter sleeve 34 would accommodate a 28/37 liner to form a liner/sleeve subassembly that could be positioned in the shell, again leaving 2 mm of metal for the wall thickness of sleeve 34.

As another example, if an existing liner design e.g. 32/39, is shifted outward in its shell to provide an offset, space becomes available between the liner and the shell to accommodate an adapter sleeve to seat a new 32/39 liner in the shell. Perhaps with stronger ceramic composites, a 32/37 or 32/38 liner would be possible in the future.

Thus it is apparent that using my adapter sleeve, a surgeon has the option of

1.) using the same ceramic liner design in which case there is no extra room in the shell for the sleeve other than that which is created in the design;

2.) using a ceramic liner with the same internal diameter but with a smaller outer diameter than that of the original liner, thus leaving room for the sleeve from the start, and

3.) downsizing the diameter of the femoral head or bearing 12a (FIG. 1) which would allow the inner diameter of the liner to be smaller. This downsizing is easy to accomplish with present day ceramic prostheses because the femoral stem has modular head and neck components which can be changed during revision so that a new ceramic head can be fitted on a new tapered neck. However, this option is the least desirable because replacement hips with smaller diameter bearings are easier to dislocate.

In FIGS. 3 and 4 and the examples just described, the inner and outer surfaces 34a and 34b of the adapter sleeve 34 are parallel to one another and the outer diameter of sleeve 34 is substantially the same as the outer diameter of the liner 18 (FIG. 2) which it replaced. Therefore, the center of curvature C′ in FIG. 4 is at more or less the same location as the center of curvature C in FIG. 2, typically within the liner at the planes P, P′. However in some cases, it may be desirable to offset the center of curvature C′ of the cup revision from the center of curvature C of the original cup shown in FIG. 2. For this, an adapter sleeve may be used which takes up more space than the original liner 18 being replaced.

A cup revision assembly such as this is shown in FIG. 5. As seen there, the assembly includes an adapter sleeve 42 whose outer dimension enables it to seat in the shell 16. Sleeve 42 has a wall thickness which is greater than that of sleeve 34 in FIG. 4. Therefore, if the assembly should include the same liner 32 shown in FIG. 4, the liner would be displaced outward (i.e. downward in FIG. 5) relative to shell 16. In other words, plane P′ would be offset from plane P and the liner's center of curvature C′ would be shifted relative to the original center C in the same direction, i.e. perpendicular to plane P as shown in FIG. 5, typically in the order of 3-4 mm. Not only would this increase in sleeve wall thickness produce a stronger adapter sleeve, since the center of curvature C′ is moved perpendicular to the shell plane P, a hip fitted with this cup revision assembly would have a higher offset relative to the previous position of the hip replacement.

Offset is the total horizontal width of the hip reconstruction relative to the preoperative state and may be increased by placing an offset liner or by using a femoral stem that has a neck with a larger horizontal dimension. Since these acetabular components are typically inserted at about a 45° angle, having a liner where the center of rotation C is translated outward would equally increase offset and leg length by the same amount. The net effect of both of these changes is to tighten the soft tissues around the hip, rendering it more stable, and, therefore, the tissues surrounding the replacement would be tighter. This is desirable in a situation where the original hip replacement is loose or is being revised because of recurrent dislocation of the hip.

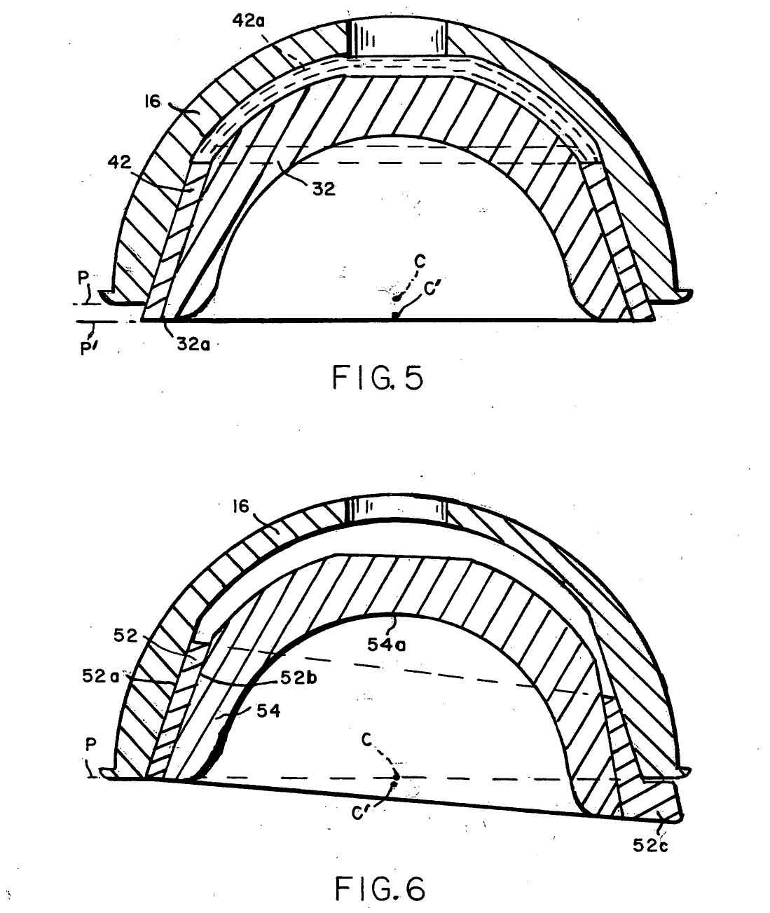

FIG. 6 illustrates another cup revision assembly which when placed in a shell, such as shell 16, results in a tilting of the liner relative to the shell. As shown in that figure, this assembly comprises an adapter sleeve 52 whose outer and inner surfaces 52a and 52b, respectively, are not parallel to one another. While the outer surface 52a is tapered to lie flush against the seat 16a of shell 16 in FIGS. 2, 4 and 5, the tapered inner surface 52b of the sleeve is tilted in one direction or another relative to surface 52a. In other words, the axes of curvature of sleeve surfaces 52a and 52b are angularly offset from one another. This results in a sleeve whose wall, in cross section, is wedge shaped and varies in thickness around the sleeve axis. Resultantly, when a liner 54 is seated in the sleeve, the liner socket 54a becomes tilted relative to the shell 16. This causes both a lateral displacement i.e. to the left in plane P, and a vertical offset, i.e. perpendicular to plane P, of the socket's center of curvature C′ which shifts are exaggerated in FIG. 6 for clarity. This can be advantageous because in some hip replacements, the socket is not oriented in the precisely correct position which can cause high wear, an impingement or a dislocation of the hip. In this situation, if the shell 16 is well fixed, the problem can be alleviated or solved by performing a revision with an adapter sleeve 52 that tilts the liner 54 by a few degrees, e.g. up to 10°. This tilting may be done in combination with the offset described above so that there is enough room to place an adapter sleeve whose wall is sufficiently thick to last for a prolonged period.

With a tilted liner as shown in FIG. 6 whatever part of the sleeve rim that is exposed beyond the rim of shell 52 is preferably thickened as shown at 52b for increased strength and to better support the liner, so long as the thickened rim 52b does not “bottom-out” on the shell rim preventing proper wedging of the sleeve into the shell. For the same reason, the exposed rim of the liner 42 in the offset assembly shown in FIG. 5 may be thickened in the same manner.

Thus the present invention enables efficient revision of the acetabular cup component of a total hip replacement. The revision can be accomplished with a minimum time and effort and with minimum likelihood of damage to the cup's liner that might necessitate a further revision of the hip replacement. The adapter sleeve of my assembly may be designed to fit a variety of standard acetabular cup shells and liners to provide a revision whose socket center of curvature has the same location as that of the original cup or a different location to suit the particular circumstances.

It will thus be seen that the objects set forth above among those made apparent from the preceding description are efficiently attained and, since certain changes may be made in carrying out the above method and in the constructions set forth without departing from the scope of the invention. For example, instead of the adapter sleeve and liner being separate parts which are assembled by the surgeon, in some cases they could is be premanufactured as a unit at the factory. This would enable the use of various manufacturing techniques to improve the strength and quality of the assembly, e.g. heatshrinking the sleeve around the liner. Also, the adapter sleeve could have a closed interior end that fits between the shell and the liner as shown in phantom at 42a in FIG. 5. Such a sleeve would help to support the liner 32 rendering the liner less likely to fracture. In a sense, that sleeve would be equivalent to a new or honed seat surface to replace the old disfigured surface of an existing shell to create a new smooth interface for a new ceramic liner. Therefore, it is intended that all matter contained in the above description or shown in the accompanying drawings be interpreted as illustrative and not in a limiting sense.

It is also to be understood that the following claims are intended to cover all of the generic and specific features of the invention described herein.

1. An acetabular cup assembly comprising
a cup-like liner, said liner having a rim defining a first plane, an inner surface defining a hemispherical socket with a center of curvature substantially at the first plane and an outer frustoconical seating surface extending to said rim, said seating surface having a first taper, and
a sleeve having inner and outer frustoconical surfaces each with an axis of curvature and surrounding said liner, the inner surface of said sleeve having the same taper as said seating surface so that the sleeve wedges against said seating surface to fix the position of said center of curvature relative to the sleeve and the outer surface of the sleeve having a selected second taper that matches that of a cup shell seat surface.

2. The assembly defined in claim 1 wherein the sleeve is of metal.

3. The assembly defined in claim 2 wherein the liner is of a ceramic.

4. The assembly defined in claim 3 wherein the metal is a titanium alloy or cobalt chromium alloy and the ceramic is alumina or a combination of alumina and zirconia.

5. The assembly defined in claim 1 wherein the inner and outer surfaces of the sleeve are parallel to one another.

6. The assembly defined in claim 1 wherein the axes of said inner and outer surfaces are coincident.

7. The assembly in claim 1 wherein the axes of said inner and outer surfaces are angularly offset.

8. The assembly defined in claim 1 wherein the sleeve includes a closed interior end extension which overlies the liner.

9. The assembly defined in claim 1 and further including a generally hemispherical shell surrounding said sleeve, said shell having a rim defining a second plane and an inner frustoconical seat surface extending into said shell from said second plane, said seat surface having the same taper as that of said outer surface so that the sleeve wedges against said seat surface to fix the position of said center of curvature relative to the shell.

10. The assembly defined in claim 9 wherein first and second tapers are the same.

11. The assembly defined in claim 9 wherein the first and second tapers are different.

12. The assembly defined in claim 1 wherein the spacing of the inner and outer surfaces of the sleeve is uniform.

13. The assembly defined in claim 1 wherein the spacing of the inner and outer surfaces of the sleeve varies.

14. An adapter sleeve for wedging between the shell and lining of an acetabular cup assembly, said sleeve including a wall having radially inner and outer frustoconical surfaces and opposite ends, each surface having an axis of curvature.

15. The adapter sleeve defined in claim 14 wherein said wall has a uniform thickness.

16. The adapter sleeve defined in claim 14 wherein said axes of curvature are coincident.

17. The adapter sleeve defined in claim 14 wherein said axes of curvature are parallel but laterally offset from one another.

18. The adapter sleeve defined in claim 14 wherein said axes are angularly offset from one another.

19. The adapter sleeve defined in claim 14 wherein said inner and outer surfaces have the same conical taper.

20. The adapter sleeve defined in claim 14 wherein the inner and outer surfaces have different conical tapers.

21. The adapter sleeve defined in claim 14 and further including a closed end extension integral to one end of the sleeve.

22. A method of revising an implanted acetabular cup of the type including a cup-like outer shell having a rim and an inner seat surface extending from the rim into the shell, said seat surface having a selected first conical taper, and an inner cup-like liner having a circular rim with an outer diameter, an inner surface defining a hemispherical socket with a center of curvature and an outer frustoconical seating surface extending to the rim, said seating surface having substantially the same conical taper as said seat surface, said liner being positioned in the shell so that the seating surface of the liner is in wedging engagement with the seat surface of the shell, said method comprising the steps of
surgically accessing the acetabular cup;
removing the liner from the shell while the shell remains in place;
providing a cup-like replacement liner having a circular rim with a selected outer diameter that is smaller than the outer diameter of the rim of said liner, an outer wall defining a seating surface and an inner wall defining a hemispherical socket with a center of curvature;
providing a frustoconical adjuster sleeve having an inner surface with the same taper as that of the seating surface of the replacement liner, an outer surface having the same taper as that of the shell seat and a selected wall thickness between said surfaces;
inserting the replacement liner into the shell so that the outer surface of the replacement liner is in flush, wedging engagement with the inner surface of the sleeve;
inserting the adjuster sleeve into said shell so that the outer surface of the adjuster sleeve is in flush, wedging engagement with the shell seat, and
surgically closing the access to said cup.

23. The method defined in claim 22 including providing a metal sleeve and a ceramic replacement liner.

24. The method defined in claim 22 wherein claim 22 wherein said inner and outer surfaces are parallel to one another.

25. The method defined in claim 24 including providing a replacement liner having a rim whose outer diameter plus twice the wall thickness of the adjuster sleeve is substantially equal to the outer diameter of the rim of said liner.

26. The method defined in claim 22 wherein the inner and outer surfaces of the adapter sleeve have coincident axes of curvature.

27. The method defined in claim 22 wherein said inner and outer surfaces have axes of curvature which are angularly offset from one another.

28. The method defined in claim 22 including forming the adjuster sleeve with an integral closed interior end that extends between the shell and the liner.

29. The method defined in claim 22 wherein said selected thickness of the adjuster sleeve is sufficient to essentially resurface the seat surface of the shell to accommodate the replacement liner.
